(19) United States
(12) Patent Application Publication
(10) Pub. No.: US 2026/0354798 A1
(43) Pub. Date: Nov. 12, 2026

(54) ULTRASOUND DIAGNOSTIC DEVICE AND METHOD

(71) Applicant: SAMSUNG MEDISON CO., LTD., Hongcheon-gun, Gangwon-do (KR)

(72) Inventors: Mo Se Kim, Hongcheon-gun, Gangwon-do (KR); Yoon Chang Lee, Hongcheon-gun, Gangwon-do (KR)

(73) Assignee: SAMSUNG MEDISON CO., LTD., Hongcheon-gun, Gangwon-do (KR)

(21) Appl. No.: 19/125,665

(22) PCT Filed: May 10, 2023
(86) PCT No.: PCT/KR2023/006383

§ 371 Date: Apr. 29, 2025

(30) Foreign Application Priority Data
Jan. 16, 2023 (KR) 10-2023-0006372

(51) Int. Cl. A61B 8/00

(52) U.S. Cl. CPC A61B 8/481; A61B 8/5269

(57) ABSTRACT

An ultrasound diagnosis method according to an embodiment of the present disclosure includes a step of scanning a contrast-enhanced image for a part of interest of an object after entering a contrast-enhanced image mode, a step of transmitting an ultrasound signal for destroying a contrast agent, and a step of accumulating re-perfused contrast-enhanced images, and further includes a step of adjusting a PRI according to reflection artifact caused by a non-part of interest adjacent to the part of interest.

### TECHNICAL FIELD

The present disclosure relates to ultrasound diagnostic apparatuses and diagnosis methods, and more particularly, to an ultrasound diagnostic apparatus and diagnosis method capable of automatically adjusting a pulse interval.

### BACKGROUND ART

An ultrasound image refers to a result of sending sound waves of a high frequency from the surface of a human body to the inside of the human body and then imaging sound waves reflected by the inside. Ultrasound examination provides the ultrasound image in real time.

Ultrasound diagnostic apparatuses transmit an ultrasound signal generated by a transducer of a probe to an object and receive information of an echo signal reflected by the object, thereby obtaining an image of an internal part of the object. These ultrasound diagnostic apparatuses have advantages, including stability and real-time display, and safety, compared to X-ray diagnosis apparatuses, and thus, the ultrasound diagnostic apparatuses are commonly used together with other image diagnosis apparatuses. In particular, ultrasound diagnostic apparatuses are safe because they are more accurate than other diagnosis apparatuses and t there is no risk of radiation exposure to a human body, and accordingly are widely used in various diagnostic processes.

Ultrasound contrast agents (UCAs) are used in ultrasound diagnostic apparatuses. UCAs are injected into an object to enhance a contrast between tissues, thereby providing more precise ultrasound images.

However, the accuracy of ultrasound images decreases depending on presence or absence of a strong reflector located behind an object. To address this problem, technology development is needed.

### DISCLOSURE OF INVENTION

### Technical Problem

The purpose of the present disclosure is to provide an ultrasound diagnostic apparatus and diagnosis method for increasing the accuracy of ultrasound images, by controlling a pulse repetition interval (PRI) according to whether rear artifact of an object is caused during a process of using ultrasound contrast agents.

### Solution to Problem

An ultrasound diagnosis method according to the present disclosure includes a step of scanning a contrast-enhanced image(or contrast agent image) for a part of interest of an object after entering a contrast-enhanced image mode, a step of transmitting an ultrasound signal for destroying a contrast agent, and a step of accumulating re-perfused contrast-enhanced images, and further includes a step of adjusting a PRI according to reflection artifact caused by a non-part of interest adjacent to the part of interest.

In detail, the step of adjusting the PR may be followed by the step of transmitting the ultrasound signal for destroying the contrast agent.

In detail, the step of transmitting the ultrasound signal for destroying the contrast agent may be followed by the step of adjusting the PR.

In detail, the step of adjusting the PRI may include a step of determining the reflection artifact, and a step of adjusting the PRI according to the reflection artifact.

In detail, the step of determining the reflection artifact may include a step of obtaining first image data to which the first PRI, which is a PRI set when the contrast-enhanced image mode is set, has been applied, a step of obtaining second image data to which the second PRI with an increased pulse interval compared with the first PRI has been applied, a step of performing a difference operation on the first image data and the second image data, and a step of determining whether a result of the difference operation is equal to or greater than a preset threshold.

In detail, the step of performing the difference operation may be at least one of a difference between the pieces of image data, a correlation analysis, or a neural network.

In detail, the ultrasound diagnosis method may further include determining a location where the reflection artifact occurs.

In detail, the ultrasound diagnosis method may further include adaptively adjusting the PRI according to the determined location of the reflection artifact.

In detail, in the setting of the contrast-enhanced image mode, the PRI may be set.

In detail, the view depth may be fixed.

In detail, the step of scanning the contrast-enhanced image may include scanning a contrast-enhanced image of the object by applying the second PRI having an increased pulse interval compared with the pre-set first PRI.

In detail, after the accumulation of the contrast-enhanced image is completed, the PRI may be maintained as the second PRI or restored to the first PRI.

In detail, the step of adjusting the PRI may include, when the result of the difference operation is less than the threshold, adjusting the PRI to the first PRI, and, when the result of the difference operation is greater than or equal to the threshold, maintaining the second PRI.

In detail, the step of scanning the contrast-enhanced image may further include a step of scanning a contrast-enhanced image of an object by applying the second PRI having an increased pulse interval compared with the preset first PRI, and the step of determining the reflection artifact may include a step of obtaining third image data to which a third PRI, which is a PRI set when the contrast-enhanced image mode is set, has been applied, a step of obtaining second image data to which the second PRI with an increased pulse interval compared with the third PRI has been applied, a step of performing a difference operation on the third image data and the second image data, and a step of determining whether a result of the difference operation is equal to or greater than a preset threshold. In the step of adjusting the PRI, when the result of the difference operation is less than the threshold, the PRI may be adjusted to the third PRI, and when the result of the difference operation is greater than or equal to the threshold, the PRI may be adjusted to the second PRI.

In detail, the setting of the contrast-enhanced image mode may include an operation of varying the PRI when a signal for changing a view depth is input.

In detail, the ultrasound diagnosis method may include a step of switching to a contrast agent destruction mode.

In the step of switching to the contrast agent destruction mode, when an input signal is input through a control panel or a touch panel, the ultrasound diagnostic apparatus may be switched to the contrast agent destruction mode.

In detail, in the step of destroying the contrast agent, an ultrasound signal may be applied to the object for a preset period of time with a negative pressure at which the contrast agent is destroyed.

An ultrasound diagnosis method according to the present disclosure includes a step of varying a PRI when a contrast agent imaging mode is entered and a signal for changing a view depth is input, a step of scanning a contrast-enhanced image of an object, a step of transmitting an ultrasound signal for destroying a contrast agent, and a step of accumulating re-perfused contrast-enhanced images.

In detail, the step of varying the PRI according to the view depth may include a step of adjusting the view depth while the PRI is being fixed, and a step of increasing the PRI when lowering the view depth, and decreasing the PRI when increasing the view depth.

In detail, the ultrasound diagnosis method may further include a step of obtaining a fifth image according to a fifth PRI increased from the fourth PRI in a state where the view depth is adjusted, a step of obtaining a sixth image according to a sixth PRI decreased from the fifth PRI, a step of performing a difference operation between the fifth image data and the sixth image data, a step of determining whether a result of the difference operation is greater than or equal to a preset threshold, and a step of, when the result of the difference operation is less than the threshold, maintaining the sixth PRI, and, when the result of the difference operation is greater than or equal to the threshold, adjusting the PRI to the fifth PRI.

In detail, in the step of varying the PRI according to the view depth, the PRI may vary according to the view depth.

In detail, in the step of varying the PRI according to the view depth, different PRIs may be set according to different view depths.

In detail, a PRI may be adjusted according to reflection artifact caused by a non-part of interest adjacent to a part of interest of the object.

In detail, the step of adjusting the PRI may include a step of determining the reflection artifact of the object, and a step of adjusting the PRI according to the reflection artifact.

In detail, the step of scanning the contrast-enhanced image may include scanning a contrast-enhanced image of the object by applying the second PRI having an increased pulse interval compared with the pre-set first PRI.

In detail, the step of determining the reflection artifact may include a step of obtaining first image data to which the first PRI, which is a PRI set when the contrast-enhanced image mode is entered, has been applied, a step of obtaining second image data to which the second PRI with an increased PRI, a step of performing a difference operation on the first image data and the second image data, and a step of determining whether a result of the difference operation is equal to or greater than a preset threshold.

In detail, the step of adjusting the PRI may include, when the result of the difference operation is less than the threshold, adjusting the PRI to the first PRI, and, when the result of the difference operation is greater than or equal to the threshold, maintaining the second PRI.

In detail, the ultrasound diagnosis method may further include a step of scanning a contrast-enhanced image of an object by applying the second PRI having an increased pulse interval compared with the preset first PRI, and the step of determining the reflection artifact may include a step of obtaining third image data to which a third PRI, which is a PRI set when the contrast-enhanced image mode is set, has been applied, a step of obtaining second image data to which the second PRI with an increased pulse interval compared with the third PRI has been applied, a step of performing a difference operation on the third image data and the second image data, and a step of determining whether a result of the difference operation is equal to or greater than a preset threshold. In the step of adjusting the PRI, when the result of the difference operation is less than the threshold, the PRI may be adjusted to the third PRI, and when the result of the difference operation is greater than or equal to the threshold, the PRI may be adjusted to the second PRI.

In detail, the ultrasound diagnosis method may include a step of switching to a contrast agent destruction mode.

In the step of switching to the contrast agent destruction mode, when an input signal is input through a control panel or a touch panel, the ultrasound diagnostic apparatus may be switched to the contrast agent destruction mode.

In detail, in the step of destroying the contrast agent, an ultrasound signal may be applied to the object for a preset period of time with a negative pressure at which the contrast agent is destroyed.

An ultrasound diagnostic apparatus according to the present disclosure includes a scanning unit for scanning a contrast-enhanced image for a part of interest of an object after a contrast-enhanced image mode is entered, a contrast agent destruction unit for transmitting an ultrasound signal for destroying a contrast agent, a control unit for determining reflection artifact due to a non-part of interest adjacent to the part of interest and adjusting a PRI according to the reflection artifact, and an image accumulation unit for accumulating re-perfused contrast-enhanced images.

In detail, the scanning unit and the contrast agent destruction unit may share a transducer.

In detail, in the determination of the reflection artifact, first image data to which the first PRI, which is a PRI set when the contrast-enhanced image mode is set, has been applied, and second image data to which the second PRI with an increased pulse interval compared with the first PRI has been applied may be obtained, a difference operation may be performed on the first image data and the second image data, and it may be determined whether a result of the difference operation is equal to or greater than a preset threshold.

In detail, in the adjustment of the PRI, when the result of the difference operation is less than the threshold, the PRI may be adjusted to the first PRI, and, when the result of the difference operation is greater than or equal to the threshold, the second PRI may be maintained.

In detail, the PRI may vary according to view depths.

In detail, different PRIs may be set according to different view depths.

In detail, in the varying of the PRI according to the view depth, the view depth may be adjusted while the PRI is being fixed, and the PRI may be increased when the view depth is lowered, and the PRI may be decreased when the view depth is increased.

### Advantageous Effects of Invention

An ultrasound diagnostic apparatus and diagnosis method according to the present disclosure enables a pulse repetition interval (PRI) to be adjusted according to occurrence of rear artifact, thereby increasing the accuracy of ultrasound images.

The effects of the present disclosure are not limited to the aforementioned effects, and effects not mentioned will be clearly understood by those of ordinary skill in the art to which the disclosure belongs from the present specification and the accompanying drawings.

### BRIEF DESCRIPTION OF DRAWINGS

FIG. 1 is a block diagram of an ultrasound diagnostic apparatus 100 according to an embodiment of the present disclosure.

FIG. 2 is a block diagram of a structure of an ultrasound diagnostic apparatus 100 according to an embodiment of the present disclosure.

FIG. 3 is a block diagram of a structure of an ultrasound diagnostic apparatus 100 according to an embodiment of the present disclosure.
(a) through (c) of FIG. 4 are perspective views of an ultrasound diagnostic apparatus 200 according to at least one embodiment of the present disclosure.
(a) through (c) of FIG. 5 are perspective views of ultrasound diagnostic apparatuses 500 according to one or more embodiments of the present disclosure.

FIG. 6 is a drawing for explaining different images according to sizes of a Frame Rate (FR).

FIG. 7 is a view for explaining occurrence of reflection artifact due to the rear of an object.

FIG. 8 is a view for explaining a diagnosis method using a conventional ultrasound diagnostic apparatus.

FIG. 9 is a view for explaining a diagnosis method performed by an ultrasound diagnostic apparatus 700 according to an embodiment of the present disclosure.

FIG. 10 is a view for explaining a method of determining whether rear artifact is caused, in a diagnosis method performed by an ultrasound diagnostic apparatus 700 according to an embodiment of the present disclosure.

FIG. 11 is a view for explaining a diagnosis method using a conventional ultrasound diagnostic apparatus.

FIG. 12 is a view for explaining a diagnosis method performed by an ultrasound diagnostic apparatus 700 according to another embodiment of the present disclosure.

FIG. 13 is a block diagram of an ultrasound diagnostic apparatus 700 according to an embodiment of the present disclosure.

### MODE FOR THE INVENTION

The principle of the present disclosure is explained and embodiments are disclosed so that the scope of the present disclosure is clarified and one of ordinary skill in the art to which the present disclosure pertains implements the present disclosure. The disclosed embodiments may have various forms.

Throughout the specification, when an element is referred to as being “connected” or “coupled” to another element, it can be directly connected or coupled to the other element, or can be indirectly connected or coupled to the other element. The indirect connection or coupling includes connection or coupling via a wireless communication network.

The terms used in the present specification are merely used to describe particular embodiments, and are not intended to limit and/or restrict the disclosed invention. An expression used in the singular encompasses the expression of the plural, unless it has a clearly different meaning in the context. In the present specification, the terms such as “including”, “having”, and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

While such terms as “first”, “second”, etc., may be used to describe various components, such components must not be limited to the above terms. The above terms are used only to distinguish one component from another. For example, a first component discussed below could be termed a second component, and similarly, a second component may be termed a first component without departing from the teachings of the present disclosure.

The terms “unit”, “-er (-or)”, “block”, “member”, and “module” may refer to a unit in which at least one function or operation is performed. For example, the above terms may refer to at least one hardware, such as a field-programmable gate array (FPGA)/application specific integrated circuit (ASIC), at least one software stored in a memory, or at least one process processed by a processor.

Symbols respectively attached to steps are used to distinguish between the steps, and do not indicate the order of the steps. The steps may be performed in a different order than the stated order, unless the context clearly indicates a specific order.

An image used herein may include a medical image captured by a medical imaging apparatus, such as a magnetic resonance imaging (MRI) apparatus, a computed tomography (CT) apparatus, an ultrasound imaging apparatus, or an X-ray apparatus, and an ultrasound image and a medical image having a different modality from ultrasound may also be provided or controlled.

Throughout the specification, a term ‘object’ is a thing to be imaged, and may include a human, an animal, or a part of a human or animal. For example, the object may include a part of a body (i.e., an organ), a phantom, or the like.

Throughout the specification, an “ultrasound image” refers to an image of an object processed based on ultrasound signals transmitted to the object and reflected therefrom.

An embodiment according to the present disclosure will now be described more fully with reference to the accompanying drawings.

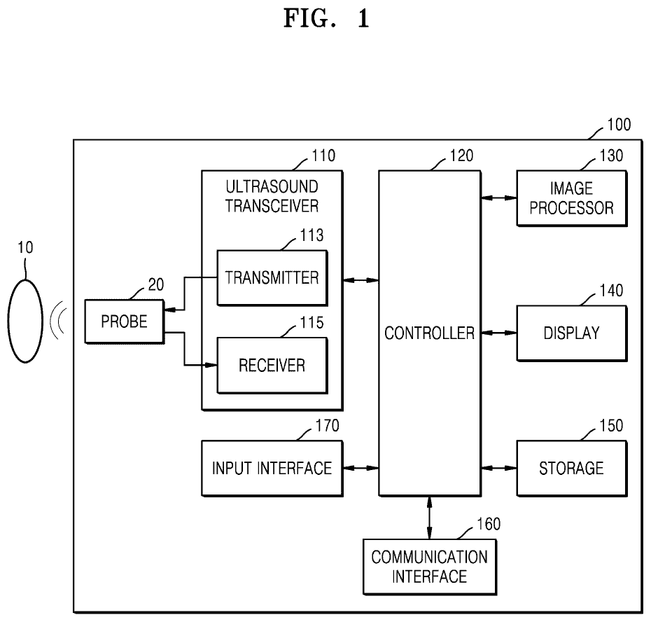

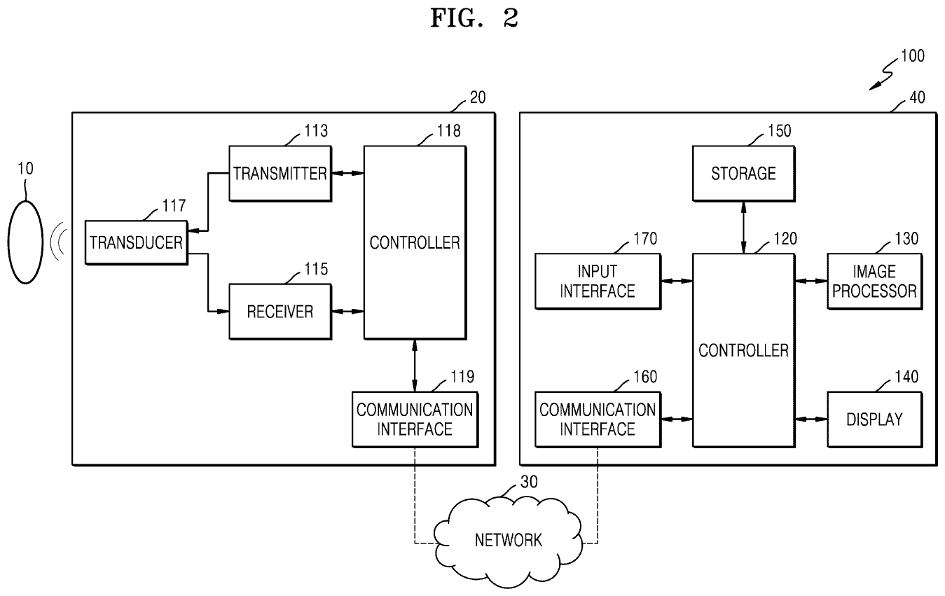

FIG. 1 is a block diagram of an ultrasound diagnostic apparatus 100 according to an embodiment of the present disclosure.

The ultrasound diagnostic apparatus 100 according to an embodiment may include a probe 20, an ultrasound transceiver 110, a controller 120, an image processor 130, a display 140, a storage 150, a communication interface 160, and an input interface 170.

The ultrasound diagnostic apparatus 100 may be implemented as not only a cart type ultrasound diagnostic apparatus but also as a portable ultrasound diagnostic apparatus. Examples of the portable ultrasound diagnostic apparatus may include, but are not limited to, a smartphone, a laptop computer, a personal digital assistant (PDA), or a tablet PC each including a probe and an application.

The probe 20 may include a plurality of transducers. The plurality of transducers may transmit ultrasound signals to an object 10 according to a transmission signal applied by a transmitter 113. The plurality of transducers may receive ultrasound signals reflected by the object 10 and may form reception signals. The probe 20 may be integrated with the ultrasound diagnostic apparatus 100, or may be separate from the ultrasound diagnostic apparatus 100 and may be connected thereto via a wired-wireless communication. In addition, the ultrasound diagnostic apparatus 100 may include one probe 20 or a plurality of probes 210 according to embodiments.

The controller 120 controls the transmitter 113 to form a transmission signal that is to be applied to each of the plurality of transducers, considering respective positions and respective focal points of the plurality of transducers included in the probe 20.

The controller 120 may control the receiver 115 to generate ultrasound data by performing analog-to-digital conversion on the reception signals received from the probe 20 and summing digital reception signals by considering the respective positions and focus points of the plurality of transducers.

The image processor 130 generates an ultrasound image by using ultrasound data generated by an ultrasound receiver 115.

Meanwhile, the ultrasound image may be represented as not only a grayscale ultrasound image obtained by scanning an object in an amplitude (A) mode, a brightness (B) mode, and a motion (M) mode, but also as a Doppler image showing movement of the object.

The A mode is a most basic form among ultrasound image display methods. The A mode is a method of displaying the intensity of reflected sound as an amplitude size on a time (distance) axis. When a reflected sound is strong, the amplitude is high, and when the reflected sound is weak, the amplitude is low. Thus, the A mode is advantageous for distance measurement, but it is currently rarely used because an image changes even when the direction of a probe is slightly different.

The M mode is a modified form of the A mode, and is a mode of displaying a distance of a moving reflector as a temporal change. The M mode is to designate a region of interest (ROI) in a two-dimensional (2D) image as an M line and display changes in the region over time, and thus is mainly used to observe heart valves. The M mode may also record fetal heart sounds, but it is recently being replaced by a Doppler method.

The B mode is a method of displaying reflected sounds as the brightness of dots, and is currently used in most ultrasound diagnosis equipment. In the B mode, the brightness of each dot is proportional to the amplitude of a reflection signal. Recently, the B mode is a mode of providing a brightness level of 256 or greater and imaging an organ movement in real time. A mode called a 2D mode refers to a brightness (B) mode, is a mode of displaying a cross-section image of an object on the screen in black and white shades in real time, and is a most commonly used mode.

The Doppler mode is a mode of generally measuring a blood flow by detecting a flow of red blood cells in blood vessel, and uses a principle that the wavelength becomes shorter when the red blood cells approach the probe and the wavelength becomes longer when the red blood cells move away from the probe. According to methods of displaying a blood flow, there are a color Doppler, a pulse wave Doppler (PW), a continuous wave Doppler (CW), etc. The Doppler image may include a blood flow Doppler image showing a flow of blood (or also referred to as a color Doppler image), a tissue Doppler image showing a movement of tissue, and a spectral Doppler image showing a moving speed of an object as a waveform.

In addition, examples of a composite mode include a mode of applying two or three modes to one image at the same time to display other modes together with the 2D mode by using the 2D mode as a base, and a three-dimensional (3D) mode of displaying a three-dimensional stereoscopic image.

In a B mode processing process, a B mode component is extracted from ultrasound data and processed, and, in an image generation process, an ultrasound image indicating signal intensities as brightness may be generated based on the extracted B mode components. In a Doppler processing process, Doppler components may be extracted from ultrasound data, and, in the image generating process, a Doppler image indicating a movement of the object 10 as colors or waveforms may be generated based on the extracted Doppler components.

In the image generating process, a 2D or 3D ultrasound image of an object may be generated, and an elasticity image which visualizes deformation of the object 10 due to a pressure may also be generated. Furthermore, several pieces of additional information may be displayed on an ultrasound image by using text and graphics. The generated ultrasound image may be stored in a memory.

In a process of measuring an object in an ultrasound image, a measuring tool for measuring the object may be determined, and one of a plurality of measuring tools may be selected based on a user input.

For example, a measurement tool selection menu for selecting one of a plurality of measuring tools may be provided, and a measuring tool selection menu may be displayed on a single screen together with an ultrasound image. In addition, the measuring tool selection menu may be displayed on a separate screen different from a touch screen on which the ultrasound image is displayed.

Moreover, one of the plurality of measuring tools may be determined based on a user input of selecting one from a plurality of measuring items that are to be measured. The measuring items may include, but are not limited to, a length, a width, or an angle.

In response to the user input of selecting one from the measuring items, a predetermined measuring tool may be determined in correspondence with the selected measuring item.

The display 140 may display the generated ultrasound image, and various pieces of information processed by the ultrasound diagnostic apparatus 100. The ultrasound diagnostic apparatus 100 may include one or more displays 140 according to embodiments. The display 140 may be coupled with a touch panel and thus may be implemented as a touch screen.

The controller 120 may control all operations of the ultrasound diagnostic apparatus 100 and a signal flow between the internal components of the ultrasound diagnostic apparatus 100. The controller 120 may include a memory storing programs or data for performing functions of the ultrasound diagnostic apparatus 100, and a processor configured to process the programs or the data. The controller 120 may receive a control signal from the input interface 170 or an external device to control an operation of the ultrasound diagnostic apparatus 100.

The ultrasound diagnostic apparatus 100 may include the communication interface 160, and may be connected to the external device (for example, a server, a medical apparatus, and a portable device (smartphone, tablet personal computer (PC), or wearable device)), via the communication interface 160.

The communication interface 160 may include one or more components that enable communication with the external device. For example, the communication interface 160 may include a short distance communication module, a wired communication module, and a wireless communication module.

The communication interface 160 may transmit and receive a control signal and data from the external device, and may transmit the received control signal to the controller 120 so that the controller 120 may control the ultrasound diagnostic apparatus 100 according to the received control signal.

Alternatively, the controller 120 may transmit a control signal to the external device via the communication interface 160 so that the external device may be controlled according to the control signal of the controller.

For example, the external device may process the data of the external device according to the control signal of the controller received via the communication interface.

Because a program (artificial intelligence, etc.) capable of controlling the ultrasound diagnostic apparatus 100 may be installed on the external device, the program may include instructions for performing some or all of the operations of the controller 120.

The program may be pre-installed on the external device, or a user of the external device may download the program from a server providing an application, and may install the downloaded program. The server providing an application may include a recording medium having the program recorded thereon.

The program may include, in a system including a server and a client device, a storage medium of the server or a storage medium of the client device. Alternatively, when there is a third device (e.g., a smartphone, a tablet PC, or a wearable device) in communication with the server or client device, a program product may include a storage medium of the third device. Alternatively, the program may include a software (S/W) program itself transmitted from the server to the client device or the third device, or transmitted from the third device to the client device.

In this case, one of the server, the client device, and the third device may execute the program to perform methods according to disclosed embodiments. Alternatively, at least two of the server, the client device, and the third device may execute the program to distribute and perform the methods according to the disclosed embodiments.

For example, a server (e.g., a cloud server or an artificial intelligence server) may execute a program stored in the server to control the client device communicating with the server to perform the methods according to the disclosed embodiments.

The storage 150 may store various data or programs for driving and controlling the ultrasound diagnostic apparatus 100, input/output ultrasound data, an obtained ultrasound image, etc.

The input interface 170 may receive a user input for controlling the ultrasound diagnostic apparatus 100. For example, the user input may include, but is not limited to, an input for manipulating a button, a key pad, a mouse, a trackball, a jog switch, a knop, or the like, an input for touching a touch pad or a touch screen, a voice input, a motion input, and a biometric data input (e.g., iris recognition and fingerprint recognition).

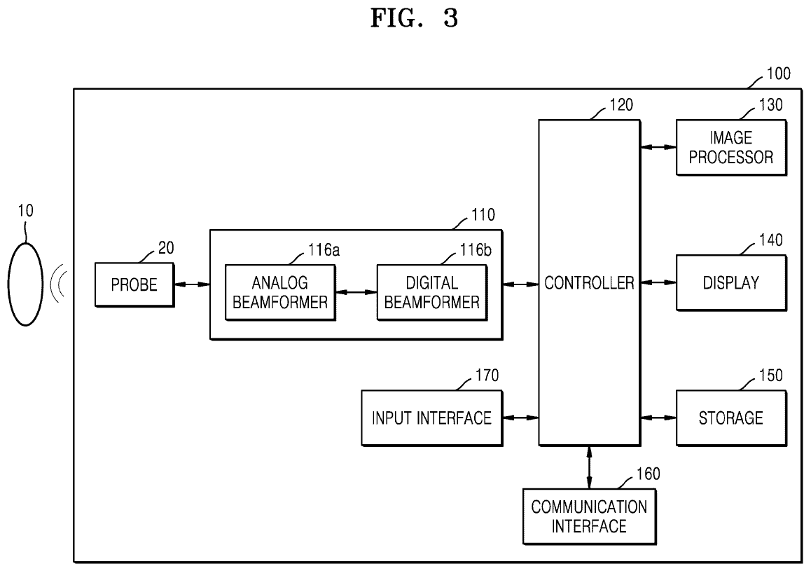

FIG. 2 is a block diagram of a structure of an ultrasound diagnostic apparatus 100 according to an embodiment of the present disclosure.

Referring to FIG. 2, the ultrasound diagnostic apparatus 100 may include a wireless probe 20 and an ultrasound system 40.

The wireless probe 20 may include a transmitter 113, a transducer 117, a receiver 115, a controller 118, and a communication interface 119. In FIG. 2, the wireless probe 20 is shown as including both the transmitter 113 and the receiver 115. However, according to embodiments, the wireless probe 20 may include only some of the transmitter 113 and the receiver 115, or some of the transmitter 113 and the receiver 115 may be included in the ultrasound system 40. Alternatively, the wireless probe 20 may further include an image processor 130.

The transducer 117 may include a plurality of transducers. The plurality of transducers may transmit ultrasound signals to the object 10 according to a transmission signal applied by the transmitter 113. The plurality of transducers may receive ultrasound signals reflected by the object 10 and may form reception signals.

The controller 118 controls the transmitter 113 to form a transmission signal that is to be applied to each of the plurality of transducers, considering respective positions and respective focal points of the plurality of transducers.

The controller 118 may control the receiver 115 to generate ultrasound data by performing analog-to-digital conversion on the reception signal received from the transducer 117 and summing digital reception signals by considering the respective positions and focus points of the plurality of transducers. Alternatively, when the wireless probe 20 includes the image processor 130, the wireless probe 20 may generate an ultrasound image by using the generated ultrasound data.

The communication interface 119 may wirelessly transmit the generated ultrasound data or ultrasound image to the ultrasound system 40 through a wireless network. Alternatively, the communication interface 119 may receive a control signal and data from the ultrasound system 40.

In addition, the ultrasound diagnostic apparatus 100 may include one or more wireless probes 20 according to embodiments.

The ultrasound system 40 may receive the ultrasound data or the ultrasound image from the wireless probe 20. The ultrasound system 40 may include a controller 120, an image processor 130, a display 140, a storage 150, a communication interface 160, and an input interface 170.

The image processor 130 generates an ultrasound image by using the ultrasound data received from the wireless probe 20.

The display 140 may display the ultrasound image received from the wireless probe 20, the ultrasound image generated by the ultrasound system 40, and various pieces of information that are processed by the ultrasound diagnostic apparatus 100. The ultrasound diagnostic apparatus 100 may include one or more displays 140 according to embodiments. The display 140 may be coupled with a touch panel and thus may be implemented as a touch screen.

The controller 120 may control all operations of the ultrasound diagnostic apparatus 100 and a signal flow between the internal components of the ultrasound diagnostic apparatus 100. The controller 120 may include a memory storing programs or data for performing functions of the ultrasound diagnostic apparatus 100, and a processor configured to process the programs or the data. The controller 120 may receive a control signal from the input interface 170 or an external device to control an operation of the ultrasound diagnostic apparatus 100.

The ultrasound system 40 includes the communication interface 160, and may be connected to external devices, such as a server, a medical apparatus, and a portable device (smartphone, tablet PC, wearable device, etc.), via the communication interface 160.

The communication interface 160 may include one or more components that enable communication with the external device. For example, the communication interface 160 may include a short distance communication module, a wired communication module, and a wireless communication module.

The communication interface 160 may transmit and receive a control signal and data from the external device, and may transmit the received control signal to the controller 120 so that the controller 120 may control the ultrasound diagnostic apparatus 100 according to the received control signal.

Alternatively, the controller 120 may transmit a control signal to the external device via the communication interface 160 so that the external device may be controlled according to the control signal of the controller.

For example, the external device may process the data of the external device according to the control signal of the controller received via the communication interface.

Because a program (artificial intelligence, etc.) capable of controlling the ultrasound diagnostic apparatus 100 may be installed on the external device, the program may include instructions for performing some or all of the operations of the controller 120.

The program may be pre-installed on the external device, or a user of the external device may download the program from a server providing an application, and may install the downloaded program. The server providing an application may include a recording medium having the program recorded thereon.

The program may include, in a system including a server and a client device, a storage medium of the server or a storage medium of the client device. Alternatively, when there is a third device (e.g., a smartphone, a tablet PC, or a wearable device) in communication with the server or client device, a program product may include a storage medium of the third device. Alternatively, the program may include a S/W program itself transmitted from the server to the client device or the third device, or transmitted from the third device to the client device.

In this case, one of the server, the client device, and the third device may execute the program to perform methods according to disclosed embodiments. Alternatively, the client device may perform the methods according to the disclosed embodiments via the server.

Alternatively, at least two of the server, the client device, and the third device may execute the program to distribute and perform the methods according to the disclosed embodiments.

For example, a server (e.g., a cloud server or an artificial intelligence server) may execute a program stored in the server to control the client device communicating with the server to perform the methods according to the disclosed embodiments.

The storage 150 may store various data or programs for driving and controlling the ultrasound diagnostic apparatus 100, input/output ultrasound data, an ultrasound image, etc.

The input interface 170 may receive a user input for controlling the ultrasound diagnostic apparatus 100. For example, the user input may include, but is not limited to, an input for manipulating a button, a key pad, a mouse, a trackball, a jog switch, a knop, or the like, an input for touching a touch pad or a touch screen, a voice input, a motion input, and a biometric data input (e.g., iris recognition and fingerprint recognition).

FIG. 3 is a block diagram of a structure of an ultrasound diagnostic apparatus 100 according to an embodiment of the present disclosure.

Referring to FIG. 3, the ultrasound diagnostic apparatus 100 may include a probe 20, an ultrasound transceiver 110, a controller 120, an image processor 130, a display 140, an input interface 170, a storage 150, and a communication interface 160.

The probe 20 according to an embodiment may include a plurality of transducers. The plurality of transducers may be arranged in a 2D configuration to form a 2D transducer array.

For example, the 2D transducer array may include a plurality of sub-arrays arranged in a second direction different from a first direction and each including a plurality of transducers arranged in the first direction.

The ultrasound transceiver 110 may include an analog beamformer 116a and a digital beamformer 116b. In FIG. 3, the ultrasound transceiver 110 and the probe 20 are illustrated as separate components. However, the probe 20 according to an embodiment may include some or all of the components of the ultrasound transceiver 110 according to embodiments. For example,
the probe 20 may include one of the analog beamformer 116a and the digital beamformer 116b, or may include both of them.

The controller 120 may calculate a time delay value for digital beamforming for each sub-array, with respect to each of the plurality of sub-arrays included in the 2D transducer array. The controller 120 may calculate a time delay value for analog beamforming with respect to each of the transducers included in one of the plurality of sub-arrays.

The controller 120 may control the analog beamformer 116a and the digital beamformer 116b to form a transmission signal that is to be applied to each of the plurality of transducers, according to the time delay value for analog beamforming and the time delay values for digital beamforming.

The controller 120 may control the analog beamformer 116a to sum signals received from the plurality of transducers for each sub-array according to the time delay values for analog beamforming. The controller 120 may also control the ultrasound transceiver 110 to perform analog-to-digital conversion with respect to a signal corresponding to a result of the summation for each sub-array. The controller 120 may control the digital beamformer 116b to generate ultrasound data, by summing digital signals according to the time delay values for digital beamforming.

The image processor 130 generates an ultrasound image by using the generated ultrasound data.

The display 140 may display the generated ultrasound image, and various pieces of information processed by the ultrasound diagnostic apparatus 100. The ultrasound diagnostic apparatus 100 may include one or more displays 140 according to embodiments. The display 140 may be coupled with a touch panel and thus may be implemented as a touch screen.

The controller 120 may control all operations of the ultrasound diagnostic apparatus 100 and a signal flow between the internal components of the ultrasound diagnostic apparatus 100. The controller 120 may include a memory storing programs or data for performing functions of the ultrasound diagnostic apparatus 100, and a processor configured to process the programs or the data. The controller 120 may receive a control signal from the input interface 170 or an external device to control an operation of the ultrasound diagnostic apparatus 100.

The ultrasound diagnostic apparatus 100 may include the communication interface 160, and may be connected to the external device (for example, a server, a medical apparatus, and a portable device (smartphone, tablet personal computer (PC), or wearable device)), via the communication interface 160.

The communication interface 160 may include one or more components that enable communication with the external device. For example, the communication interface 160 may include a short distance communication module, a wired communication module, and a wireless communication module.

The communication interface 160 may transmit and receive a control signal and data from the external device, and may transmit the received control signal to the controller 120 so that the controller 120 may control the ultrasound diagnostic apparatus 100 according to the received control signal.

Alternatively, the controller 120 may transmit a control signal to the external device via the communication interface 160 so that the external device may be controlled according to the control signal of the controller.

For example, the external device may process the data of the external device according to the control signal of the controller received via the communication interface.

Because a program (artificial intelligence, etc.) capable of controlling the ultrasound diagnostic apparatus 100 may be installed on the external device, the program may include instructions for performing some or all of the operations of the controller 120.

The program may be pre-installed on the external device, or a user of the external device may download the program from a server providing an application, and may install the downloaded program. The server providing an application may include a recording medium having the program recorded thereon.

The program may include, in a system including a server and a client device, a storage medium of the server or a storage medium of the client device. Alternatively, when there is a third device (e.g., a smartphone, a tablet PC, or a wearable device) in communication with the server or client device, a program product may include a storage medium of the third device. Alternatively, the program may include a S/W program itself transmitted from the server to the client device or the third device, or transmitted from the third device to the client device.

In this case, one of the server, the client device, and the third device may execute the program to perform methods according to disclosed embodiments. Alternatively, at least two of the server, the client device, and the third device may execute the program to distribute and perform the methods according to the disclosed embodiments.

For example, a server (e.g., a cloud server or an artificial intelligence server) may execute a program stored in the server to control the client device communicating with the server to perform the methods according to the disclosed embodiments.

The storage 150 may store various data or programs for driving and controlling the ultrasound diagnostic apparatus 100, input/output ultrasound data, an ultrasound image, etc.

The input interface 170 may receive a user input for controlling the ultrasound diagnostic apparatus 100. For example, the user input may include, but is not limited to, an input for manipulating a button, a key pad, a mouse, a trackball, a jog switch, a knop, or the like, an input for touching a touch pad or a touch screen, a voice input, a motion input, and a biometric data input (e.g., iris recognition and fingerprint recognition).
(a) through (c) of FIG. 4 are perspective views of an ultrasound diagnostic apparatus 200 according to at least one embodiment of the present disclosure.

Referring to (a) and (b) of FIG. 4, ultrasound diagnostic apparatuses 200a and 200b may include a main display 221 and a sub-display 222. One of the main display 221 and the sub-display 222 may be implemented by using a touch screen. The main display 221 and the sub-display 222 may display an ultrasound image, or various pieces of information that are processed by the ultrasound diagnostic apparatuses 200a and 200b. The main display 221 and the sub-display 222 may be implemented by using touch screens, and may provide graphical user interfaces (GUIs) to receive data for controlling the ultrasound diagnostic apparatuses 200a and 200b from a user. For example, the main display 221 may display the ultrasound image, and the sub-display 222 may display a control panel for controlling display of the ultrasound image, in a GUI form. The sub-display 222 may receive data for controlling display of an image, via the control panel displayed in a GUI form. The ultrasound diagnostic apparatuses 200a and 200b may control display of an ultrasound image displayed on the main display 221, by using the received control data.

Referring to (b) of FIG. 4, the ultrasound diagnostic apparatus 200b may further include a control panel 165 in addition to the main display 221 and the sub-display 222. The control panel 265 may include buttons, a trackball, a jog switch, a knob, etc., and may receive data for controlling the ultrasound diagnostic apparatus 200b from the user. For example, the control panel 265 may include a Time Gain Compensation (TGC) button 271, a Freeze button 272, etc. The TGC button 271 is a button for setting a TGC value for each depth of the ultrasound image. When an input of the Freeze button 272 is detected while the ultrasound image is being scanned, the ultrasound diagnostic apparatus 200b may maintain the display of a frame image at the point of time, capture the frame image at that point of time.

The buttons, the trackball, the jog switch, the knob, etc. included in the control panel 265 may be provided as a GUI on the main display 221 or sub-display 222.

Referring to (c) of FIG. 4, an ultrasound diagnostic apparatus 200c may also be implemented by using a portable ultrasound diagnostic apparatus. Examples of the portable ultrasound diagnostic apparatus 200c may include, but are not limited to, a smartphone, a laptop computer, a PDA, or a tablet PC each including a probe and an application.

The ultrasound diagnostic apparatus 200c may include the probe 20 and a main body 240. The probe 20 may be connected to one side of the main body 240 by wire or wirelessly The main body 240 may include a touch screen 245. The touch screen 245 may display, for example, an ultrasound image, various pieces of information processed by an ultrasound diagnostic apparatus, and a GUI.
(a) through (c) of FIG. 5 are perspective views of ultrasound diagnostic apparatuses 500 according to one or more embodiments of the present disclosure.

Referring to (a) of FIG. 5, an ultrasound diagnosis device used indoors or indoor ultrasound diagnostic apparatus 500 refers to a non-portable ultrasound diagnostic apparatus generally used for ultrasound diagnosis, and such an ultrasound diagnostic apparatus 500 is also referred to as cart-based equipment. The ultrasound diagnostic apparatus 500 does not necessarily need to be used only indoors. However, for convenience, the ultrasound diagnostic apparatus 500 will be referred to as an indoor ultrasound diagnostic apparatus 500.

The indoor ultrasound diagnostic apparatus 500 may have a portable docking unit 580 that is connected to a portable ultrasound diagnostic apparatus 400, and all components of the indoor ultrasound diagnostic apparatus 500 used in the present disclosure except for the portable docking unit 580 are generally used components, so detailed descriptions thereof are omitted.

Unlike the portable ultrasound diagnostic apparatus 400, the indoor ultrasound diagnostic apparatus 500 has fewer constraints in terms of size, weight, power consumption, etc., and thus may be developed with a variety of diagnostic items and high performance. When the portable ultrasound diagnostic apparatus 400 is mounted on this indoor ultrasound diagnostic apparatus 500, the portable ultrasound diagnostic apparatus 400 may be used with high performance. However, a location where the portable ultrasound diagnostic apparatus 400 is mounted on the indoor ultrasound diagnostic apparatus 500 is not limited to any place as long as it is a location where it is convenient for a user to use the portable ultrasound diagnostic apparatus 400 and the indoor ultrasound diagnostic apparatus 500 simultaneously, and is not limited by (a) of FIG. 5. Furthermore, the portable ultrasound diagnostic apparatus 400 may be connected to the indoor ultrasound diagnostic apparatus by wire or by being mounted to and connected with the indoor ultrasound diagnostic apparatus 500.

Referring to (a) and (b) of FIG. 5, the portable ultrasound diagnostic apparatus 400 in (a) of FIG. 5 may correspond to a portable ultrasound diagnostic apparatus 201 in (b) of FIG. 5.

The portable ultrasound diagnostic apparatus 400 may be formed integrally with a probe (not shown) including a plurality of transducer elements. In detail, the portable ultrasound diagnostic apparatus 400 refers to an apparatus that provides an ultrasound image to a user by using received ultrasound image data by being connected to the indoor ultrasound diagnostic apparatus 500 by using a wireless or wired communication method (including a universal serial bus (USB)). For example, the portable ultrasound diagnostic apparatus 400 may be a smart apparatus that downloads and installs an application on a smartphone, etc.

In detail, the portable ultrasound diagnostic apparatus 400 may provide an ultrasound image to a user by using received ultrasound image data, the data being received by connecting to the indoor ultrasound diagnostic apparatus 500 via a wired or wireless communication method.

For example, the wireless communication method may include at least one of short-range data communication methods including a 60 GHz (mmWave) wireless local area network (WLAN). The wireless communication method may be a wireless local area network (Wi-Fi), Bluetooth, Zigbee, Wi-Fi Direct (WFD), Infrared Data Alliance (IrDA), Bluetooth Low Energy (BLE), Near Field Communication (NFC), Wireless Broadband Internet (Wibro), a Worldwide Interoperability Shared Wireless Access Protocol (SWAP) for Microwave Access (WiMAX), Wireless Gigabit Alliance (WiGig), or radio frequency (RF) communication.
(b) of FIG. 5 illustrates an ultrasound diagnostic system in which the portable ultrasound diagnostic apparatus 201 is connected to a cart-based ultrasound diagnostic apparatus 500.

The cart-based ultrasound diagnostic apparatus 500 may be connected to the portable ultrasound diagnostic apparatus 201 by using the aforementioned wireless communication method. In detail, the portable ultrasound diagnostic apparatus 201 may include at least one wireless communication module (not shown) for performing at least one of the aforementioned wireless communication methods. Furthermore, the portable docking unit 580 of the cart-based ultrasound diagnostic apparatus 500 may include at least one wireless communication module (not shown) for performing wireless communication with the portable ultrasound diagnostic apparatus 201.

In this case, the wireless communication module in the cart-based ultrasound diagnostic apparatus 500 may be a module for performing communication according to at least one of the aforementioned wireless communication methods.
(c) of FIG. 5 illustrates an ultrasound diagnostic system in which a portable ultrasound diagnostic apparatus 202 is connected to the cart-based ultrasound diagnostic apparatus 500.

The portable ultrasound diagnostic apparatus 202 may be coupled to a probe 301 via a probe port. The portable ultrasound diagnostic apparatus 202 may generate an ultrasound image by using an ultrasound image corresponding to an ultrasound signal received by the probe 301, and may display the ultrasound image on a display.

The cart-based ultrasound diagnostic apparatus 500 may be connected to the portable ultrasound diagnostic apparatus 202 by using the aforementioned wireless communication method. The connection between the cart-based ultrasound diagnostic apparatus 500 and the portable ultrasound diagnostic apparatus 202 via wireless communication corresponds to the connection between the cart-based ultrasound diagnostic apparatus 500 and the portable ultrasound diagnostic apparatus 201, and thus a detailed description thereof will be omitted.

An embodiment of an ultrasound diagnostic apparatus and diagnosis method applicable to at least one of the ultrasound diagnostic apparatuses mentioned above with reference to FIGS. 1 through 3 will now be described.

The present disclosure relates to an ultrasound diagnostic apparatus and diagnosis method using ultrasound contrast agents (UCAs).

UCAs used in an ultrasound diagnosis method are used to obtain a more effective ultrasound image through microbubbles with an average size of 5 μm, and use a principle in which microbubbles having a similar size to an ultrasound frequency generate ultrasound scattering due to resonance and enhance an image. An ultrasound signal reflected by microbubbles is displayed as a strong signal compared to general tissues within an ultrasound image. As described above, UCAs are injected into a bloodstream and then a strong contrast agent signal in the bloodstream is detected, whereby blood vessel information may be confirmed in more detail.

In detail, a diagnosis method using UCAs involves injecting a contrast agent made up of microbubbles into a body and then artificially destroying the microbubbles with high negative pressure, and transmitting a flash pulse as an ultrasound signal to destroy the contrast agent. Then, in the diagnosis method using UCAs, a process of re-perfusing the contrast agent is performed, a contrast image is formed by photographing the re-perfused contrast agent, and images for the process are accumulated to observe changes in images. At this time, the accumulated images are visualized at a high frame rate (FR) and used for diagnosis.

In the ultrasound diagnostic apparatus and diagnosis method according to the present disclosure, a contrast-enhanced image of an object into which a contrast agent has been injected is scanned, the contrast agent is destroyed due to transmission of flash pulses, and then re-perfused contrast-enhanced images are accumulated.

In particular, image accumulation is performed immediately after the transmission of the flash pulse, so that the spreading of the contrast agent may be clearly observed.

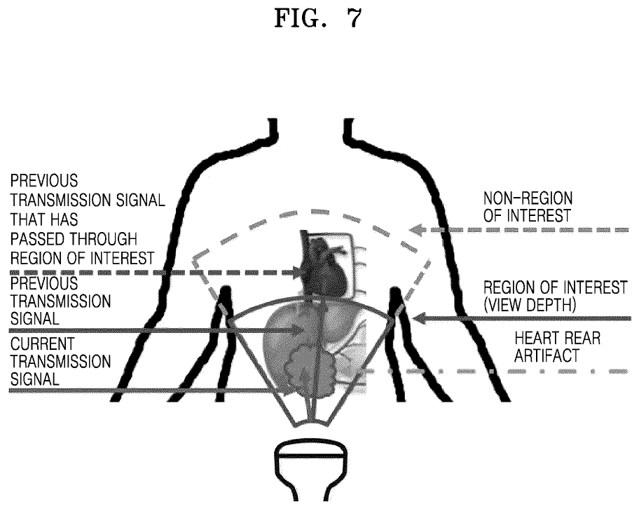

However, in the ultrasound diagnosis method using a contrast agent as described above, even when an ultrasound signal is transmitted toward an object actually corresponding to a part of interest, the ultrasound signal is transmitted to a surrounding area outside the object and reflected, resulting in generation of artifact due to a non-part of interest. This will be described below with reference to FIG. 7.

As described above, a strong rear echo signal entering by being reflected by the non-part of interest, i.e., the rear of the part of interest, appears as artifact on an image. The present disclosure relates to a technology for removing the artifact, and is characterized by automatically adjusting a pulse repetition interval (PRI) according to reflection artifacts caused by the non-part of interest located adjacent to the part of interest of the object that a user wishes to diagnose.

The PRI refers to a time interval between pulses for the same spatial location as the PRI. According to the present disclosure, the PRI according to presence or absence of reflection artifact, thereby allowing contrast agent destruction through flash pulses to occur while suppressing occurrence of a rear echo signal, and allowing a contrast agent destruction process and an image accumulation process to occur in succession.

In general, as the PRI increases, rear artifact is eliminated, but a frame rate (FR) decreases.

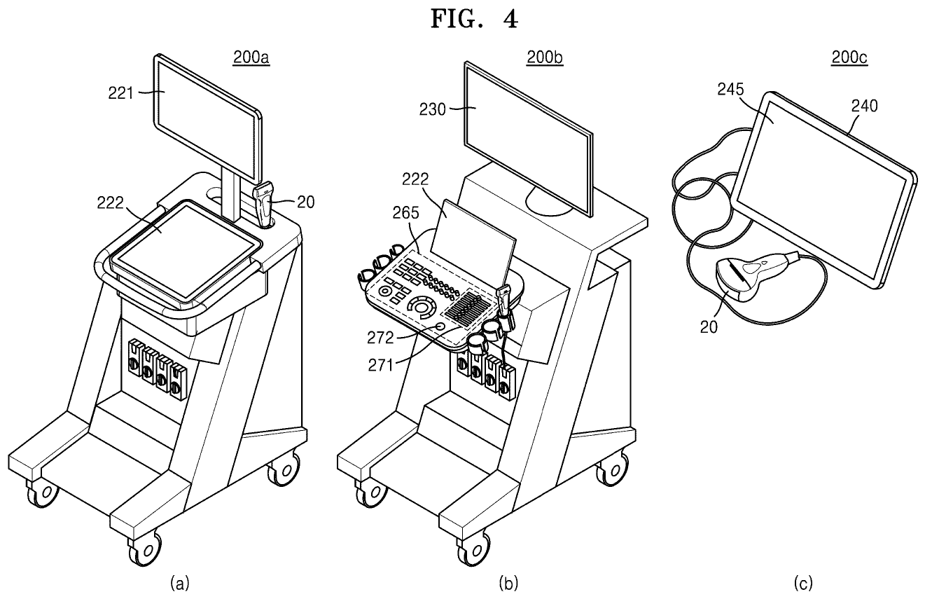

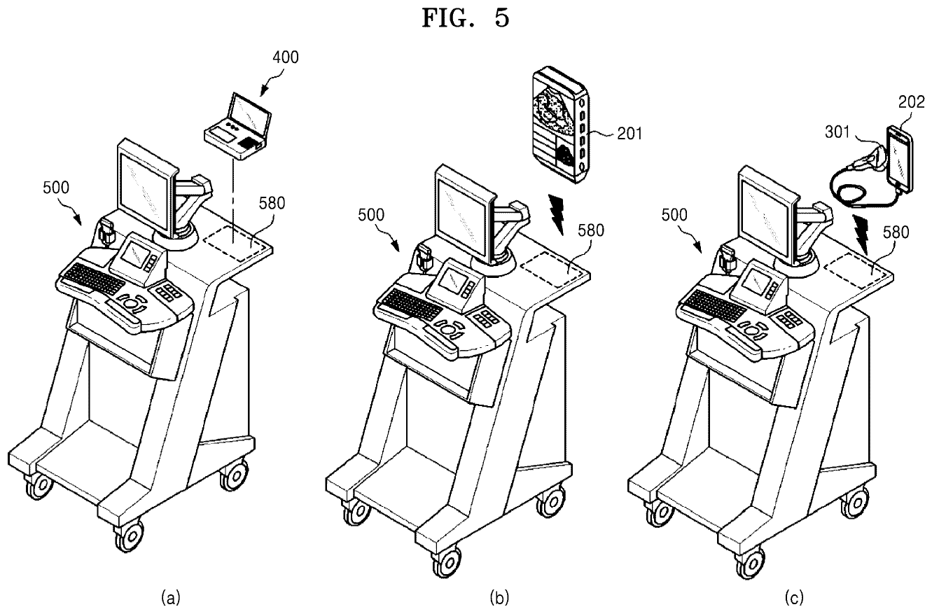

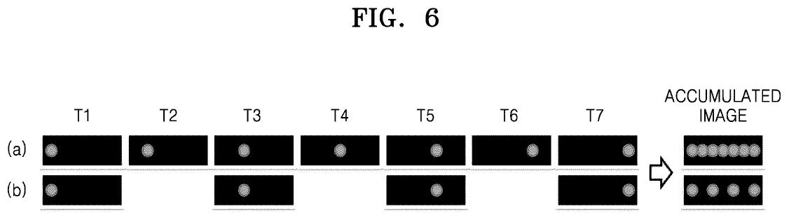

FIG. 6 is a drawing for explaining different images according to sizes of the FR.

Regarding what is shown in FIG. 6, a circular shape shown on each screen is a contrast agent bubble. The screens are depicted as T1 through T7 over time and are shown as moving to the right, and each image shown on the right corresponds to an accumulated image.

FIG. 6(a) shows an image with a high Frame Rate (FR) with a large number of frames per time, and FIG. 6(b) shows an image with a low Frame Rate (FR).

When FIG. 6(a) is compared with FIG. 6(b), it may be confirmed that connectivity and expressive power of an image are significantly superior in the image shown in FIG. 6(a) with a high Frame Rate (FR).

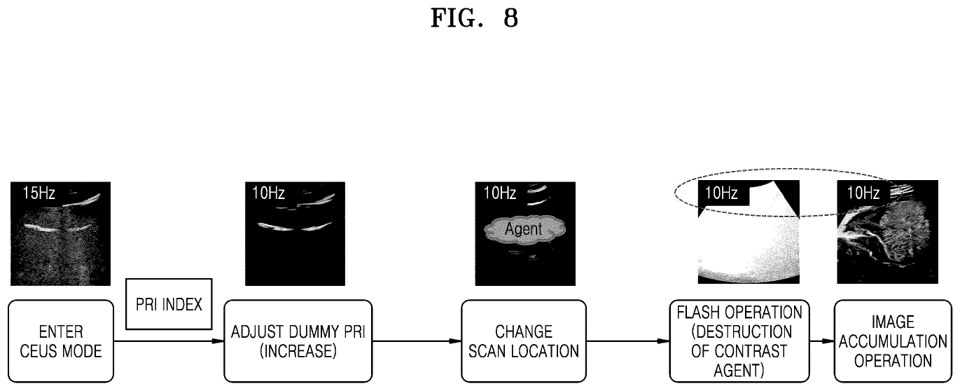

When the PRI is increased to remove rear artifact, loss of the Frame Rate (FR) is large, and thus an image quality deteriorates. In particular, as will be explained below with reference to FIG. 8, in the case of a conventional technology of unconditionally increasing a PRI (applying a dummy PRI) even when there is no strong reflector at the rear of an object, the Frame Rate (FR) inevitably decreases, leading to deterioration of the image quality.

The present disclosure is a technology of automatically determining whether rear artifact of an object occurs before increasing the PRI (applying a dummy PRI), and then appropriately applying an increase in the PRI (dummy PRI) according to a result of the determination, thereby minimizing the loss of the frame rate (FR) according to a state of the object and a state of the surroundings of the object to thereby address the deterioration of the image quality.

FIG. 7 is a view for explaining occurrence of reflection artifact due to the rear of an object.

In FIG. 7, the inside of a solid line area depicts a part of interest, i.e., a region of interest (view depth), of the object, and a dotted line area beyond the solid line area depicts a non-part of interest adjacent to the part of interest, i.e., a non-region of interest.

While a previous transmission signal shown as a solid line in FIG. 7 is passing through the region of interest, imaging is performed from a previously transmitted reflection signal, and, when the previous transmission signal leaves the region of interest and reaches the non-region of interest, a current transmission signal is generated in a probe, and imaging is performed from a currently transmitted reflection signal. At this time, a more previous transmission signal shown in a dotted line in FIG. 7 reaches the heart located in the non-region of interest, and thus a heart reflection signal is generated. At this time, the heart reflection signal overlaps with a reflection signal caused by the current transmission signal, and thus appears as heart rear artifact in an image.

As described above, a reflection signal scattered in the region of interest according to a new transmission signal temporally coincides with a signal entering by being reflected by the non-region of interest from the immediately preceding transmission signal, so that they appear as reflection artifacts in the image displayed based on the current transmission. Thus, in normal cases, the reflection artifact of the non-region of interest is attenuated and weakened while passing through a medium (human body), and the amount of attenuation increases as a distance increases. However, because a reflection signal due to current transmission is a short-range reflection signal, the amount of attenuation is obviously small, so it has greater signal characteristics than the reflection artifact of the non-region of interest. For this reason, the reflection artifact is not easy to observe.

For example, for a low-frequency convex probe with a center frequency of 3 MHz, a view depth of 16 cm is typically set. In this case, the attenuation rate of general soft tissue is 0.5 dB/cm/MHz, so the amount of attenuation while a 3 MHz transmission signal travels back and forth 16 cm is approximately 48 dB (0.5 dB/cm/MHz×3 MHz×16 cm×2). This is an attenuation ratio of about 1/250 on a linear scale, and is considerably low compared to a probe surface signal. This attenuation ratio does not easily appear in general anatomical structures, and, even when it appears, it is difficult to observe.

As described above, it is not easy to recognize rear artifact in the relevant field, and, even when rear artifact is recognized, the present disclosure of appropriately increasing or maintaining the PRI according to occurrence or non-occurrence of rear artifact is different from the conventional art of consistently using a method of increasing the PRI as will be explained with reference to FIG. 8 below.

FIG. 8 is a view for explaining a diagnosis method using a conventional ultrasound diagnostic apparatus.

In general, to suppress reflection artifact, a method of increasing the PRI is generally used. When a dummy PRI is added, a rear echo is reduced and thus artifact may be minimized. However, the FR decreases as the PRI increases.

FIG. 8 is a process of consistently applying a dummy PRI to minimize rear artifact, as a conventional method.

First, a contrast-enhanced image mode is entered, and then, in order to remove the rear artifact, a user applies the Dummy PRI to thereby increase the PRI (decrease the FR from 15 Hz to 10 Hz), performs a scan with the rear artifact removed, and destroys the contrast agent and accumulates images through a flash operation.

However, in the conventional method, the FR is reduced from 15 Hz to 10 Hz as the PRI is unconditionally increased even when there is no strong reflector at the rear, and there is a problem in performing a flash operation by using the reduced FR without changes. That is, as shown in FIG. 8, in the conventional technology, the FR is always lost regardless of occurrence of rear artifact, and as a result, an image speed is slowed down and thus an image inevitably appears to be cut off.

In the present disclosure, in order to address the aforementioned problem, the PRI is adaptively adjusted by automatically determining whether reflection artifact occurs due to the rear of an object, that is, a non-part of interest adjacent to the object's part of interest, thereby minimizing rear reflection artifact and at the same time minimizing the loss of the FR.

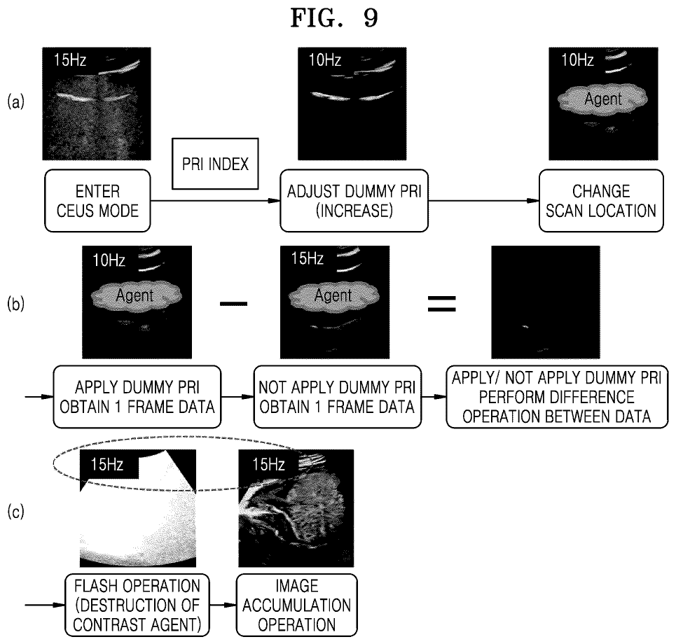

FIG. 9 is a view for explaining a diagnosis method performed by an ultrasound diagnostic apparatus according to an embodiment of the present disclosure.

Values of the PRI and the FR described below are only examples, and the PRI and the FR are not limited to these values.

As illustrated in FIG. 9, an ultrasound diagnosis method according to an embodiment of the present disclosure includes a step of scanning a contrast-enhanced image for a part of interest of an object after entering a contrast-enhanced image mode (the step (a) of FIG. 9), a step of transmitting an ultrasound signal for destroying a contrast agent, and a step of accumulating re-perfused contrast-enhanced images (the step (c) of FIG. 9), and further includes a step of adjusting a PRI according to reflection artifact caused by a non-part of interest adjacent to the part of interest (the step (c) of FIG. 9).

In detail, the step (a) of FIG. 9 illustrates a user scanning an image from which rear artifact has been removed by applying a dummy PRI, after entering a contrast-enhanced image mode. This is the same as a conventional process illustrated in FIG. 8, and the present disclosure differs from the conventional technology in a process of adjusting the PRI according to the reflection artifact caused due to the non-part of interest adjacent to the part of interest, i.e., due to the rear, illustrated in the step (b) of FIG. 9 and the step (c) of FIG. 9.

The step (b) of FIG. 9 illustrates a process of determining the reflection artifact and adjusting the PRI according to the reflection artifact, and the step (c) of FIG. 9 illustrates a process of applying the adjusted PRI to destroy the contrast agent according to a flash operation and accumulate images.

The step of accumulating the images (the step (c) of FIG. 9) in the present disclosure may be a method of adding consecutive images. However, the accumulating of the images in the present disclosure is not limited to the method of adding consecutive images. For example, a method of comparing pixel values at the same location in a previous accumulated image and a current image, and, when the pixel value of the current image is greater than the pixel value of the previous image, updating the pixel at the corresponding location to the pixel value of the current image, and, when the pixel value of the current image is less than the pixel value of the previous image, retaining the pixel value of the previous image may be used. That is, the accumulating of the images in the present disclosure may be a method in which only images that are brighter than the previous image, namely, only large pixels, are accumulated.

First, as illustrated in the step (a) of FIG. 9, in a contrast-enhanced image mode setting process in the present disclosure, the PRI may be set.

When a contrast-enhanced image mode is set, a first PRI may be initially set (FR of 15 Hz), and then a second PRI (FR of 10 Hz) with a longer pulse interval than the first PRI may be set (Dummy PRI is applied). In a step of scanning the contrast-enhanced image, a contrast-enhanced image of the object may be scanned by applying the second PRI having an increased pulse interval compared with the pre-set first PRI.

The pre-set first PRI may be considered as meaning all cases in which the PRI is shorter than the second PRI within the allowable PRI range at the image depth scanned using the second PRI.

First, the PRI according to the present disclosure may be divided into an Ideal PRI, a System PRI, and an Effective PRI. The first PRI in the present disclosure may include at least one of the Ideal PRI, the System PRI, and the Effective PRI, and the second PRI refers to a PRI value set immediately before a user executes a flash operation. Each PRI is explained through Table 1.

Assuming that ultrasound penetrates through a soft tissue at an ultrasound speed of 1540 m/s, the time it takes for the ultrasound to travel back and forth a distance of 1 cm is approximately 13 μsec. The meaning of the PRI determined at each view depth is explained below, and the PRI below is a time concept and the unit thereof is psec.

TABLE 1

| | | System | | Extra | (3) |
|---|---|---|---|---|---|
| | (1) Ideal | Dummy PRI | (2) System | Dummy PRI | Effective |
| Depth | PRI [μsec] | [μsec] | PRI [μsec] | [μsec] | PRI [μsec] |
| 5 | 65 | 13 | 78 | 156 | 234 |
| 6 | 78 | 13 | 91 | 156 | 247 |
| 7 | 91 | 13 | 104 | 156 | 260 |
| 8 | 104 | 13 | 117 | 156 | 273 |
| 9 | 117 | 13 | 130 | 156 | 286 |
| 10 | 130 | 13 | 143 | 156 | 299 |
| 11 | 143 | 13 | 156 | 156 | 312 |
| 12 | 156 | 13 | 169 | 156 | 325 |
| 13 | 169 | 13 | 182 | 156 | 338 |
| 14 | 182 | 13 | 195 | 156 | 351 |
| 15 | 195 | 13 | 208 | 156 | 364 |
| 16 | 208 | 13 | 221 | 156 | 377 |
| 17 | 221 | 13 | 234 | 156 | 390 |
| 18 | 234 | 13 | 247 | 156 | 403 |
| 19 | 247 | 13 | 260 | 156 | 416 |
| 20 | 260 | 13 | 273 | 156 | 429 |

(1) The Ideal PRI refers to an ideal time it takes for ultrasound to travel back and forth a depth of a corresponding image, that is, a depth of a part of interest scanned by a user (depth×ultrasound round-trip time), and the System Dummy PRI is defined as a time for processing, such as processing and displaying, on a monitor, of a received signal by an ultrasound system.

(2) The System PRI refers to a time corresponding to a sum of Ideal PRI and System Dummy PRI. The system PRI is a practical time in which a system dummy PRI that is delayed for signaling in a system is included in a PRI caused by a depth, and may be considered as a minimum PRI time necessary for ultrasound imaging.

For example, assuming that the System Dummy PRI is 13 μsec, a total PRI required by the 16 cm view depth is 221 μsec (208+13). The System Dummy PRI is a value that may vary according to systems, transducers, presets, and depths, but the system PRI is a value that a user cannot change, and thus is determined according to view depths.

(3) The Effective PRI refers to a final PRI obtained by including an Extra Dummy PRI, which is internally added to the system by a user or similar entity, in the system PRI. That is, the Effective PRI is a final PRI obtained by adding the same Extra Dummy PRI set by the user in the System PRI for removal of rear artifact to each depth, and is the same as the System PRI when the Extra dummy PRI is zero.

The Effective PRI is different from the System PRI that is unable to be changed by users and is determined by a depth, in that the Effective PRI is additionally set by users or engineers.

In Table 1, the Extra Dummy PRI is set to be 156 μsec. Thus, the Extra Dummy PRI is an Extra Dummy PRI necessary for preventing rear reflection artifact in a 5 cm depth when there is a reflector strong against the rear of 17 cm, and is used to create a PRI corresponding to 18 cm.

For example, when a user added Dummy PRI of 156 μsec within a system, a total PRI is 377 μsec (208+13+156), and thus 377 μsec may be considered an actual final PRI (time interval) of a part of interest of the 16 cm depth.

Referring to FIG. 9, the step (a) of FIG. 9 shows a step of scanning a contrast-enhanced image of the part of interest of the object. It may be confirmed that an artifact appears in an image obtained using the first PRI initially set after entering the contrast-enhanced image mode, and that the artifact is removed after the user adjusts the PRI to the second PRI (Extra Dummy PRI) having a longer pulse interval than the first PRI. However, when the artifact was removed, the FR was reduced from 15 Hz to 10 Hz.

In a process of scanning a contrast-enhanced image of an object to which the second PRI has been applied, a scan location may be changed to a location having no rear artifact.

The step (b) of FIG. 9 corresponds to a step of adjusting the PRI according to reflection artifact due to the non-part of interest adjacent to the part of interest, that is, due to the rear.

According to an embodiment of the present disclosure, the step of adjusting the PRI may include a step of determining the reflection artifact, and a step of adjusting the PRI according to the reflection artifact.

The step of determining the reflection artifact may include a step of obtaining first image data to which the first PRI, which is a PRI set when the contrast-enhanced image mode is set, is applied, a step of obtaining second image data to which the second PRI with an increased pulse interval compared with the first PRI is applied, a step of performing a difference operation on the first image data and the second image data, and a step of determining whether a result of the difference operation is equal to or greater than a preset threshold.

In the present disclosure, the step of performing the difference operation may use at least one of a correlation analysis and a neural network, in addition to a method of calculating a difference between the two image data, and is not limited to the listed methods.

According to an embodiment, when a negative value is obtained as a result of the difference operation, an operation of applying an absolute value to the result of the difference operation may be further performed.

In some embodiments, the steps of obtaining the first image data and the second image data may be performed consecutively without requiring separate user operations.

As shown in the step (b) of FIG. 9, the second image data (FR of 10 Hz), which is the second PRI to which the extra dummy PRI has been applied, and the first image data (FR of 15 Hz), which is the first PRI to which the extra dummy PRI has not been applied, may be individually obtained, a difference operation between the two images may be performed, and a result of the difference operation may be compared with the predetermined threshold. A value of the difference operation may be calculated as a sum of the data, or may be calculated as an average value of the data.

FIG. 9 (b) shows two images, and an image corresponding to a result of a difference operation between the two images. A threshold, which is a criterion for determining occurrence or non-occurrence of rear artifact, is a reference value that may be previously designated by a user, and thus refers to an error-level allowable value between images due to a motion, etc. The shown image corresponding to a result of a difference operation may be compared with the threshold to determine whether the result of the difference operation is less than the threshold.

According to an embodiment, in the step of adjusting the PRI, according to a result of a determination as to whether there is rear artifact of the object made based on a result value of the difference operation between the first image data, which is the first PRI, and the second image data, which is the second PRI, when the result of the difference operation is less than the threshold, the PRI may be adjusted to the first PRI, and, when the result of the difference operation is equal to or greater than the threshold, the second PRI may be maintained.

According to another embodiment of the present disclosure, the ultrasound diagnosis method may include a step of scanning a contrast-enhanced image of an object by applying the second PRI having an increased pulse interval compared with the preset first PRI, and the step of determining the reflection artifact may include a step of obtaining third image data to which a third PRI, which is a PRI set when the contrast-enhanced image mode is set, has been applied, a step of obtaining second image data to which the second PRI with an increased pulse interval compared with the third PRI has been applied, a step of performing a difference operation on the third image data and the second image data, and a step of determining whether a result of the difference operation is equal to or greater than a preset threshold.

In the step of adjusting the PRI, when a result of the difference operation between the third image data and the second image data is less than the threshold, the PRI may be adjusted to the third PRI, and, when the result of the difference operation is equal to or greater than the threshold, the PRI may be adjusted to the second PRI. The third PRI is a value having a larger PRI than the first PRI and a smaller PRI than the second PRI, and, by applying the third PRI, which is a value between the first PRI and the second PRI, the FR may be made have a larger value than when the second PRI is applied, when the result of the difference operation is equal to or greater than the threshold.

That is, while the rear artifact is minimized, the FR may be adjusted to a maximum value within a possible range.

Although not shown separately in the drawings, an image area subjected to the difference operation may be the obtained entire area, but may also be limited to the region of interest (ROI) designated by the user.

According to another embodiment, the step of determining the reflection artifact, according to the present disclosure, may further include a step of determining a location in which the reflection artifact is generated.

This is to recognize a location in which reflection artifact appears in the process of obtaining a difference operation value between the first image data and the second image data, and may further include a step of adaptively adjusting the PRI according to the determined location of the reflection artifact.

That is, in the present disclosure, the location in which the reflection artifact appears may be recognized, and, in proportion to the recognized location, the PRI may be adaptively adjusted.

The location where the reflection artifact occurs has a certain pattern. For example, when the user is viewing an image at a depth of 16 cm (part of interest) and a strong rear reflector is located at 17 cm, a rear reflection artifact may ideally appear at a depth of 1 cm, and no artifact appears beyond the position where the rear reflection artifact is located. Therefore, in the process of obtaining the difference operation value, when an image is divided at intervals of a certain section (e.g., 2 cm) and the image signals are detected only in a 0~2 cm section, even though the difference operation value is greater than the threshold and the PRI is adjusted before image accumulation, transmission would be performed using the second PRI. However, the same artifact removal effect may be obtained even when the ultrasound signal is transmitted using a third PRI, which is increased by an amount corresponding to 2 cm compared to the first PRI. In this case, when the third PRI is shorter than the second PRI, an effect of an increase in the FR also occurs. Thus, the PRI may be precisely adjusted by identifying the location of the rear reflection artifact as described above.

The step (c) of FIG. 9 corresponds to a step of transmitting an ultrasound signal for destroying the contrast agent and a step of accumulating re-perfused contrast-enhanced images.

As shown in the present disclosure, when presence or absence of rear reflection artifact is determined and the PRI is adjusted according to a result of the determination, there is no big difference in presence or absence of rear artifacts in images to which the first PRI and the second PRI have been applied, respectively. Thus, when it is determined that there is no rear artifact of an object, that is, when the difference operation value is less than the threshold, the contrast agent is destroyed at the initially-set first PRI and the images are accumulated, as in the step (c) of FIG. 9.

At this time, because the FR of the first PRI is 15 Hz and the FR of the second PRI is 10 Hz, unless artifact is finally seen large, the FR may be adjusted to 15 Hz, that is, to a maximum value, by adjusting the PRI to the first PRI, and images may be accumulated.

As a result, loss of an unnecessary FR in an object having no strong rear reflectors may be prevented, and thus a lot of images per unit time may be obtained, thereby improving the accuracy of image reading.

Compared with the present disclosure, in the method described above with reference to FIG. 8 as a conventional art, a step of transmitting an ultrasound signal for destroying the contrast agent corresponding to the step (c) of FIG. 9 and accumulating re-perfused contrast-enhanced images is performed without a step (the step (c) of FIG. 9) of adjusting the PRI according to the reflection artifact due to the non-part of interest adjacent to the part of interest.

When the PRI is increased and applied without determination of presence or absence of the rear artifact of the object, the PRI has the FR of 10 Hz as shown in FIG. 8. Thus, even when there is almost no difference between artifacts in the first PRI and the second PRI, the second PRI is applied unconditionally, and accordingly the FR is lowered. In this regard, the present disclosure (FIG. 9) by which a 15 Hz FR is maintained is significant.

In the present disclosure, the step of transmitting an ultrasound signal for destroying the contrast agent may be performed after the step of adjusting the PRI. In some embodiments, the step of adjusting the PRI may be performed after the step of transmitting an ultrasound signal for destroying the contrast agent. However, it may be more efficient to transmit an ultrasound signal for destroying the contrast agent after the step of adjusting the PRI.

In the present disclosure, after the step of accumulating the images, the FR may be restored back to the FR set when the contrast-enhanced image mode is entered, and the PRI may be maintained or restored to the second PRI, which is the PRI set when the contrast-enhanced image mode is entered. This is the second PRI that the user has finally set, and refers to a PRI having no rear artifact.

According to an embodiment, a view depth may be fixed in the contrast-enhanced image mode setting step.

In this case, the imaging process may proceed while the view depth finally set by the user immediately before the flash operation, that is, the user's ROI, is being maintained, and thus only the PRI may be adjusted. At this time, even when the PRI is adjusted, the view depth is maintained without changes.

That is, only the PRI may be changed by subtracting or adding the PRI while the view depth set by the user is being maintained, to thereby minimize an influence of the rear artifact, and respective sizes of the first image data and the second image data (a scan area and a part of interest) may not be changed.

According to another embodiment of the present disclosure, a signal for changing the view depth may be input in setting the contrast-enhanced image mode, and the contrast-enhanced image mode setting process may include a step of varying the PRI when the signal for changing the view depth is input.

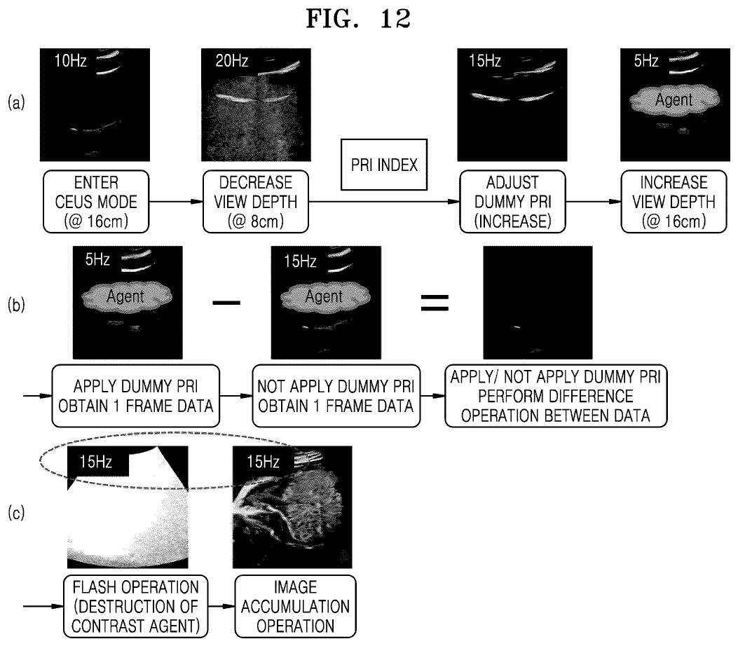

Another embodiment may include a step of varying the PRI according to a change in the view depth, and is different from the above-described embodiment in that the PRI also varies with a change in the view depth. This will be described below in detail with reference to FIG. 12.

The present disclosure may be a case where the PRI changes according to a change in the view depth, and also a case where the user adjusts the PRI while varying the PRI according to a change in the view depth.

An example for FIG. 9 will be explained with reference to Table 1 above.

When the view depth is 16 cm when a contrast agent mode is entered, the total PRI (Effective PRI) including the Extra Dummy PRI is 377 μsec, and, when the user changes the view depth to 10 cm, the total PRI (Effective PRI) is 299 μsec. When rear artifact occurs and the user applies the Extra Dummy PRI of 78 μsec, the total PRI (Effective PRI) may be 377 μsec, so the rear artifact may disappear.

When the user changes the view depth to 18 cm to observe other lesions, the total PRI (Effective PRI) becomes 481 μsec (403+78), and when the flash operation is clicked, the second image data is immediately obtained using the second PRI of 481 μsec, which is the PRI in a current state, and the first image data is also obtained using the first PRI, which does not include the Extra Dummy PRI, at the view depth of 18 cm.

A difference operation between the first image data and the second image data is calculated, and, when a result of the difference operation is greater than or equal to a preset threshold, the PRI is maintained as the second PRI, and, when the result of the difference operation is less than the threshold, the PRI is changed to the first PRI. Thereafter, the ultrasound signal for destroying the contrast agent may be transmitted, re-perfused images may be accumulated after the destruction is completed, and, when the accumulation is completed, the PRI may be changed to the second PRI at a time point just before the flash operation.

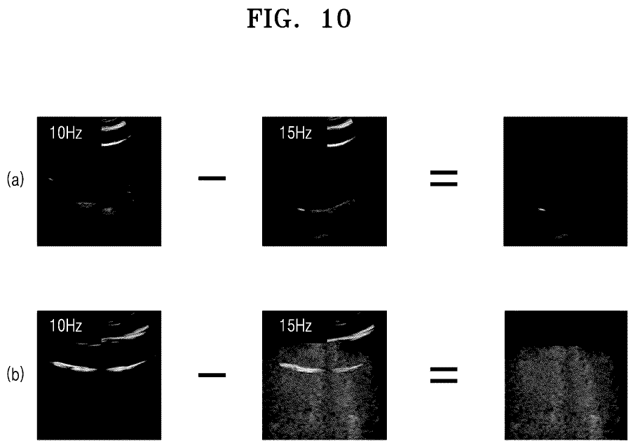

FIG. 10 is a view for explaining a method of determining whether rear artifact is present, in a diagnosis method performed by an ultrasound diagnostic apparatus according to an embodiment of the present disclosure.

FIG. 10 is a view illustrating an example of determining whether there is rear artifact, in the step of adjusting the PRI in the step (b) of FIG. 9.

On the left sides of the step (a) of an d the step (b) of FIG. 10, the second image data (Frame Rate (FR) of 10 Hz) according to the second PRI and the first image data (Frame Rate (FR) of 15 Hz) according to the first PRI are shown, respectively, and, on the right sides thereof, images resulting from difference operations between images are shown.

In the step (a) of FIG. 10, no rear artifact is visible in both the second image data and the first image data, and no rear artifact is visible in the result of the difference operation value therebetween. Thus, in this case, the difference operation value between images is less than the threshold, and thus the transmission PRI is adjusted to the first PRI. In this case, as the PRI is adjusted back to the first PRI, the Frame Rate (FR) increases to 15 Hz.

In the step (b) of FIG. 10, no rear artifact is visible in the second image data, but rear artifact is visible in the first image data, and, as a result, a result of the difference operation between the two images may be determined to be equal to or greater than a threshold. In this case, a current value of the second PRI is maintained without changes, and, as the PRI is maintained, the Frame Rate (FR) is also maintained at 10 Hz.

That is, when it is determined that there is no rear artifact, the Dummy PRI is not applied, and thus the decreased Frame Rate (FR) increases (15 Hz) again, and, when it is determined that there is rear artifact, the Dummy PRI is maintained to be applied, and thus the Frame Rate (FR) is maintained (10 Hz) without changes.

As a result, in the present disclosure, rear artifact may be removed, and the Frame Rate (FR) may be prevented from being unnecessarily lowered when there is no rear artifact.

The ultrasound diagnosis method of the present disclosure may include a step of switching to a contrast agent destruction mode, and, in the step of switching to the contrast agent destruction mode, when an input signal is input through a control panel or a touch panel, the ultrasound diagnostic apparatus is switched to the contrast agent destruction mode.

In addition, in the step of destroying the contrast agent, an ultrasound signal may be applied to the object for a preset period of time with a negative pressure at which the contrast agent is destroyed.

According to an embodiment of the present disclosure, in the step of destroying the contrast agent and the step of accumulating images, the user may set each duration through a touch panel.

Moreover, the user may set whether the image accumulation step will proceed automatically immediately after the contrast agent destruction step through option selection on the touch panel, and may also set in advance whether the determination as to whether there is rear artifact will proceed automatically or by user selection.

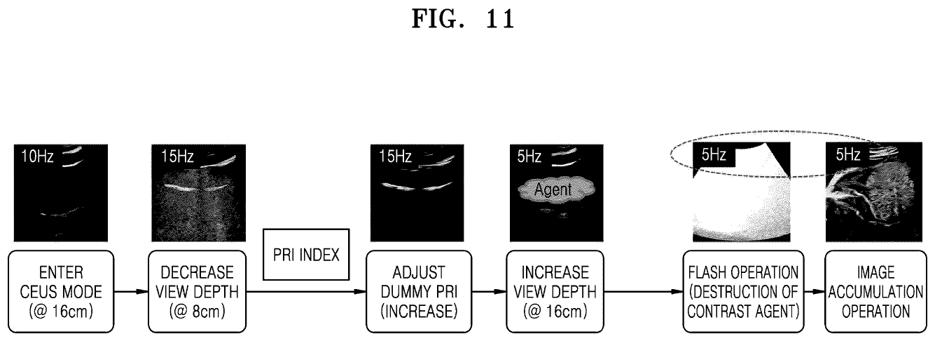

FIG. 11 is a view for explaining a diagnosis method using a conventional ultrasound diagnostic apparatus.

FIG. 11 is a view illustrating a method of performing diagnosis by adjusting a view depth, unlike FIG. 8.

According to the conventional diagnosis method illustrated in FIG. 11, after the contrast-enhanced image mode is entered, the PRI is first set to a certain value.

The view depth is initially 16 cm, but the view depth is lowered to 8 cm when the view depth is adjusted. As the view depth decreases, the Frame Rate (FR) increases (from 10 Hz to 20 Hz). During this process, rear artifact may be caused.

To remove the caused rear artifact, the user may adjust the PRI. Thus, the rear artifact may be removed by increasing the PRI. However, as the PRI increases, the Frame Rate (FR) decreases (from 20 Hz to 15 Hz).

After that, the decreased view depth may be restored back to the original view depth of 16 cm. However, in this case, the Frame Rate (FR) is lowered by 10 Hz and thus decreases to 5 Hz.

That is, the dummy PRI is applied to remove rear artifact in processes of lowering and then increasing the view depth and between the two processes, and, consequently, the frame rate (FR) is reduced, and, as shown in FIG. 11, because the images are accumulated at 5 Hz, an image speed is slowed down and thus interruption occurs.

FIG. 12 is a view for explaining a diagnosis method performed by an ultrasound diagnostic apparatus according to another embodiment of the present disclosure.

As illustrated in FIG. 12, the ultrasound diagnosis method according to another embodiment of the present disclosure may include a step of varying the PRI when a contrast agent imaging mode is entered and a signal for changing the view depth is input, a step of scanning a contrast-enhanced image of an object, a step of transmitting an ultrasound signal for destroying a contrast agent, and a step of accumulating re-perfused contrast-enhanced images.

The step of varying the PRI according to the view depth may include a step of adjusting the view depth while the PRI is being fixed, and a step of increasing the PRI when lowering the view depth, and decreasing the PRI when increasing the view depth.

Obviously, a user may set the PRI separately, apart from the PRI varying according to the view depth.

The step (a) of FIG. 12 is not a case in which, as in FIG. 11, after the contrast agent imaging mode is entered, the PRI is adjusted separately first, but rather illustrates a case where the PRI first varies according to the view depth.

The step of varying the PRI according to the view depth in the present disclosure refers to the PRI varying according to the view depth, and thus refers to a case where the PRI automatically changes according to a depth. In some cases, a user may set different PRIs according to view depths. In relation to this, Table 2 and Table 3 below will be referred to.

Referring to the step (a) of FIG. 12, the view depth is lowered, the PRI that varies according to a change in the view depth is referred to as a fourth PRI. The fourth PRI is a PRI at a view depth during scanning immediately before the flash operation after the view depth is adjusted.

Next, in order to remove rear artifact caused in the process of lowering the view depth, the dummy PRI is applied to increase the PRI. In this case, the increased PRI is referred to as a fifth PRI. In detail, the fifth PRI may be considered a PRI including, in the fourth PRI, the System Dummy PRI, and the Extra Dummy PRI added due to external inputs, such as a user and an engineer.

As in the step (a) of FIG. 12, the view depth is restored back to the original view depth of 16 cm.

The process up to the above is the same as the conventional diagnosis method, and a difference therebetween may be seen in the steps (b) and (c) of FIG. 12.

In the step (b) of FIG. 12(b), fifth image data (Frame Rate (FR) of 5 Hz) may be obtained using a fifth PRI, which has a longer pulse interval than the fourth PRI in a state where the view depth is adjusted, and a sixth image may be obtained using a sixth PRI, which has a shorter pulse interval than the fifth PRI, respectively. Also in the embodiment of FIG. 12, pieces of image data may be obtained consecutively without separate manipulation by the user.

The sixth PRI is a shorter value than the fourth PRI, and corresponds to a PRI that is longer than or equal to an Ideal PRI at which reflection artifact is minimized.

After that, a difference operation value may be obtained through a step of performing a difference operation between the fifth image data and the sixth image data, and may be compared with a pre-determined threshold to determine whether rear artifact is caused.

The step of adjusting the PRI may include a step of, according to a result of a determination as to whether there is rear artifact of the object made based on the difference operation value, maintaining the sixth PRI when the difference operation value is less than a threshold, and adjusting the PRI to the fifth PRI when the difference operation value is equal to or greater than the threshold.

The step (c) of FIG. 12 corresponds to a step of transmitting an ultrasound signal for destroying the contrast agent and a step of accumulating re-perfused contrast-enhanced images.

As shown in the step (b) of FIG. 12, when a difference operation result is less than the threshold, and there is no significant difference in presence or absence of a rear artifact in the images to which the firth PRI and the sixth PRI are applied, it is determined that there is no rear artifact of the object. In this case, as shown in the step (c) of FIG. 12, the contrast agent is destroyed at the PRI to which the dummy PRI is not applied, and images are accumulated.

Finally, unless a significant artifact is observed, images may be accumulated with a maximum Frame Rate (FR) of 15 Hz by adjusting the transmission PRI to a PRI to which the Dummy PRI is not applied.

Table 2 below explains a case of applying a variable Extra Dummy PRI to the depth, as an example in which the PRI varies according to a change in the view depth.

TABLE 2

| | | System | | Extra | |
|---|---|---|---|---|---|
| | (1) Ideal | Dummy | (2) System | Dummy | (3) Effective |
| Depth | PRI [μsec] | PRI [μsec] | PRI [μsec] | PRI [μsec] | PRI [μsec] |
| 5 | 65 | 13 | 78 | 156 | 234 |
| 6 | 78 | 13 | 91 | 143 | 234 |
| 7 | 91 | 13 | 104 | 130 | 234 |
| 8 | 104 | 13 | 117 | 117 | 234 |
| 9 | 117 | 13 | 130 | 104 | 234 |
| 10 | 130 | 13 | 143 | 91 | 234 |
| 11 | 143 | 13 | 156 | 78 | 234 |
| 12 | 156 | 13 | 169 | 65 | 234 |
| 13 | 169 | 13 | 182 | 52 | 234 |
| 14 | 182 | 13 | 195 | 39 | 234 |
| 15 | 195 | 13 | 208 | 26 | 234 |
| 16 | 208 | 13 | 221 | 13 | 234 |
| 17 | 221 | 13 | 234 | 0 | 234 |
| 18 | 234 | 13 | 247 | 0 | 247 |
| 19 | 247 | 13 | 260 | 0 | 260 |
| 20 | 260 | 13 | 273 | 0 | 273 |

As can be seen in Table 2, when the Extra Dummy PRI decreases by 13 μsec per 1 cm, the Extra Dummy PRI becomes 156 μsec at 5 cm, the Extra Dummy PRI becomes 143 μsec at 6 cm, and, when the Extra Dummy PRI is variably set to 0 up to 17 cm, the Effective PRI at all depths is the same at 234 μsec up to 17 cm.

In this case, even when there is a strong reflector at the rear of 17 cm, the Effective PRI covers up to 18 cm, so rear reflection artifact may be minimized, and, because the PRI is the same, the Frame Rate is also the same at all depths up to 17 cm. After 17 cm, the Extra Dummy PRI becomes 0, and, as the depth increases, the Effective PRI increases, and thus the Frame Rate (FR) decreases.

Table 3 below explains an example of setting the variable Extra Dummy PRI to be different from an increment in the depth.

TABLE 3

| | | System | | Extra | |
|---|---|---|---|---|---|
| | (1) Ideal | Dummy | (2) System | Dummy | (3) Effective |
| Depth | PRI [μsec] | PRI [μsec] | PRI [μsec] | PRI [μsec] | PRI [μsec] |
| 5 | 65 | 13 | 78 | 156 | 234 |
| 6 | 78 | 13 | 91 | 149 | 240 |
| 7 | 91 | 13 | 104 | 142 | 246 |
| 8 | 104 | 13 | 117 | 135 | 252 |
| 9 | 117 | 13 | 130 | 128 | 258 |
| 10 | 130 | 13 | 143 | 121 | 264 |
| 11 | 143 | 13 | 156 | 114 | 270 |
| 12 | 156 | 13 | 169 | 107 | 276 |
| 13 | 169 | 13 | 182 | 100 | 282 |
| 14 | 182 | 13 | 195 | 93 | 288 |
| 15 | 195 | 13 | 208 | 86 | 294 |
| 16 | 208 | 13 | 221 | 79 | 300 |
| 17 | 221 | 13 | 234 | 72 | 306 |
| 18 | 234 | 13 | 247 | 65 | 312 |
| 19 | 247 | 13 | 260 | 58 | 318 |
| 20 | 260 | 13 | 273 | 51 | 324 |

As can be seen in Table 3, when the Extra Dummy PRI decrease by 7 μsec per 1 cm, the PRI decrement is less than a PRI increment according to an increase in depth. In this case, the Effective PRI increases according to the depth as shown in Table 3, and an effect of adjusting the Frame Rate (FR) according to a change in depth may be maintained.

In the description related to FIG. 12, the fourth PRI, the fifth PRI, and the sixth PRI have been described in relation to a feature that the PRI varies according to a change in view depth. However, the features described above with reference to FIGS. 9 and 10, such as the step of setting the PRI, the step of determining reflection artifact, the step of adjusting the PRI, and the step of switching the ultrasound diagnostic apparatus) to the contrast agent destruction mode, may also be applicable in the process of setting the contrast-enhanced image mode.

For example, the PRI may be adjusted according to reflection artifact caused by a non-part of interest adjacent to a part of interest of an object, and the step of adjusting the PRI may include a step of determining reflection artifact of the object, and a step of adjusting the PRI according to the reflection artifact.

The step of determining the reflection artifact may include a step of obtaining first image data to which the first PRI, which is a PRI set when the contrast-enhanced image mode is entered, has been applied, a step of obtaining second image data to which the second PRI increased from the PRI has been applied, a step of performing a difference operation on the first image data and the second image data, and a step of determining whether a result of the difference operation is equal to or greater than a preset threshold. In the step of adjusting the PRI, when the result of the difference operation is less than the threshold, the PRI may be adjusted to the first PRI, and when the result of the difference operation is greater than or equal to the threshold, the second PRI may be maintained.

A step of scanning a contrast-enhanced image of an object by applying the second PRI having an increased pulse interval compared with the preset first PRI may be further included, and the step of determining the reflection artifact may include a step of obtaining third image data to which a third PRI, which is a PRI set when the contrast-enhanced image mode is set, has been applied, a step of obtaining second image data to which the second PRI with an increased pulse interval compared with the third PRI has been applied, a step of performing a difference operation on the third image data and the second image data, and a step of determining whether a result of the difference operation is equal to or greater than a preset threshold. In the step of adjusting the PRI, when the result of the difference operation is less than the threshold, the PRI may be adjusted to the third PRI, and when the result of the difference operation is greater than or equal to the threshold, the PRI may be adjusted to the second PRI.

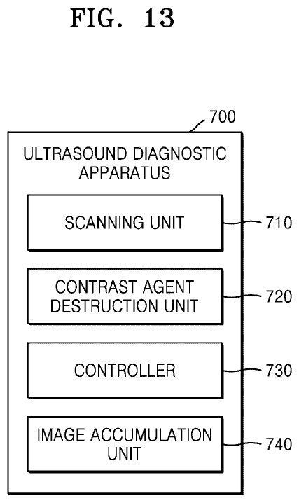

FIG. 13 is a block diagram of an ultrasound diagnostic apparatus 700 according to an embodiment of the present disclosure.

As illustrated in FIG. 13, the ultrasound diagnostic apparatus 700 according to an embodiment of the present disclosure may include a scanning unit 710 for scanning a contrast-enhanced image for a part of interest of an object after a contrast-enhanced image mode is entered, a contrast agent destruction unit 720 for transmitting an ultrasound signal for destroying a contrast agent, a controller 730 for determining reflection artifact due to a non-part of interest adjacent to the part of interest and adjusting a PRI according to the reflection artifact, and an image accumulation unit 740 for accumulating re-perfused contrast-enhanced images.

The contrast agent destruction unit 720 may destroy microbubbles forming the contrast agent introduced into the inside of the object by a mechanical action of ultrasonic sound pressure, and the contrast agent destruction unit 720 may adjust at least one of a magnitude of a transmission voltage, a frequency, a diameter of a transmission area, a pattern of the transmission area, and a focal point of the transmission area.

In addition, the scanning unit 710 and the contrast agent destruction unit 720 may share a transducer, so that the scanning unit 710 and the contrast agent destruction unit 720 may be implemented at once by a single configuration.

In the determination of the reflection artifact by the controller 730, first image data to which a first PRI, which is a PRI set when a contrast-enhanced image mode is entered, has been applied, and second image data to which a second PRI with an increased pulse interval compared with the first PRI has been applied may be obtained, a difference operation on the first image data and the second image data may be performed, and it may be determined whether a result of the difference operation is equal to or greater than a preset threshold. In the adjustment of the PRI, when the result of the difference operation is less than the threshold, the PRI may be adjusted to the first PRI, and, when the result of the difference operation is greater than or equal to the threshold, the second PRI may be maintained.

In an ultrasound diagnostic apparatus 700 according to another embodiment of the present disclosure, the PRI varies according to a view depth. In the varying of the PRI according to the view depth, the view depth may be adjusted while the PRI is being fixed, and, when the view depth is lowered, the PRI may increase, and, when the view depth is increased, the PRI may decrease.

The varying of the PRI according to the view depth refers to a case where the PRI automatically changes according to the view depth. In some cases, a user may set different PRIs according to view depths.

Because each component of the ultrasound diagnostic apparatus 700 according to the present disclosure has been sufficiently described in relation to the above-described ultrasound diagnosis method according to an embodiment of the present disclosure, a detailed description thereof will be omitted.

The embodiments of the disclosure have been particularly shown and described with reference to the accompanying drawings. However, it will be understood by those of ordinary skill in the art that various changes in form and details may be made therein without departing from the spirit and scope of the present disclosure as defined by the appended claims. The disclosed embodiments should be considered in descriptive sense only and not for purposes of limitation.

1. An ultrasound diagnosis method comprising: after entering a contrast-enhanced image mode, scanning a contrast-enhanced image of a part of interest of an object;
transmitting an ultrasound signal for destroying a contrast agent; and
accumulating re-perfused contrast agent images,
wherein the ultrasound diagnosis method further comprises adjusting a pulse repetition interval (PRI) according to reflection artifact caused by a non-part of interest adjacent to the part of interest.

2. The ultrasound diagnosis method of claim 1, wherein the adjusting of the PRI comprises:
determining the reflection artifact; and
adjusting the PRI according to the reflection artifact.

3. The ultrasound diagnosis method of claim 2, wherein the determining of the reflection artifact comprises:
obtaining first image data to which a first PRI, which is a PRI set when the contrast-enhanced image mode is set, has been applied;
obtaining second image data to which a second PRI having an increased pulse interval compared to the first PRI has been applied;
performing a difference operation between the first image data and the second image data; and
determining whether a result of the difference operation is greater than or equal to a preset threshold,
wherein the adjusting of the PRI comprises,
when the result of the difference operation is less than the threshold, adjusting the PRI to the first PRI, and, when the result of the difference operation is greater than or equal to the threshold, maintaining the second PRI.

4. The ultrasound diagnosis method of claim 3, further comprising
determining a location where the reflection artifact occurs.

5. The ultrasound diagnosis method of claim 4, further comprising
adaptively adjusting the PRI according to the determined location of the reflection artifact.

6. The ultrasound diagnosis method of claim 1, wherein the scanning of the contrast-enhanced image comprises
scanning the contrast-enhanced image of the object by applying a second PRI with an increased pulse interval compared to a preset first PRI.

7. The ultrasound diagnosis method of claim 6, wherein,
after the accumulation of the contrast-enhanced image is completed, the PRI is maintained as the second PRI or restored to the first PRI.

8. The ultrasound diagnosis method of claim 2, further comprising
scanning the contrast-enhanced image of the object by applying a second PRI with an increased pulse interval compared to a preset first PRI.
wherein the determining of the reflection artifact comprises:
obtaining third image data to which a third PRI, which is a PRI set when the contrast-enhanced image mode is set, has been applied;
obtaining second image data to which a second PRI having an increased pulse interval compared to the third PRI has been applied;
performing a difference operation between the third image data and the second image data; and
determining whether a result of the difference operation is greater than or equal to a preset threshold,
wherein the adjusting of the PRI comprises,
when the result of the difference operation is less than the threshold, adjusting the PRI to the third PRI, and, when the result of the difference operation is greater than or equal to the threshold, adjusting the PRI to the second PRI.

9. An ultrasound diagnosis method comprising: entering a contrast-enhanced image mode and varying a pulse repetition interval (PRI) when a signal for changing a view depth is input;
scanning a contrast-enhanced image of an object;
transmitting an ultrasound signal for destroying a contrast agent; and
accumulating re-perfused contrast-enhanced images.

10. The ultrasound diagnosis method of claim 9, wherein the varying of the PRI according to the view depth comprises:
adjusting the view depth while the PRI is being fixed; and
when the view depth is lowered, increasing the PRI, and, when the view depth is increased, decreasing the PRI.

11. The ultrasound diagnosis method of claim 10, further comprising:
obtaining a fifth image according to a fifth PRI increased from the fourth PRI in a state where the view depth is adjusted;
obtaining a sixth image according to a sixth PRI decreased from the fifth PRI;
performing a difference operation between the fifth image data and the sixth image data;
determining whether a result of the difference operation is greater than or equal to a preset threshold; and
when the result of the difference operation is less than the threshold, maintaining the sixth PRI, and, when the result of the difference operation is greater than or equal to the threshold, adjusting the PRI to the fifth PRI.

12. The ultrasound diagnosis method of claim 9, wherein a pulse repetition interval (PRI) is adjusted according to reflection artifact caused by a non-part of interest adjacent to a part of interest of the object.

13. The ultrasound diagnosis method of claim 10, wherein the scanning of the contrast-enhanced image comprises
scanning the contrast-enhanced image of the object by applying a second PRI with an increased pulse interval compared to a preset first PRI, and the adjusting of the PRI comprises:
determining reflection artifact of the object; and
adjusting the PRI according to the reflection artifact.

14. The ultrasound diagnosis method of claim 13, wherein the determining of the reflection artifact comprises:
obtaining first image data to which a first PRI, which is a PRI set when the contrast-enhanced image mode is entered, has been applied;
obtaining second image data to which a second PRI having an increased PRI has been applied;
performing a difference operation between the first image data and the second image data; and
determining whether a result of the difference operation is greater than or equal to a preset threshold,
wherein the adjusting of the PRI comprises,
when the result of the difference operation is less than the threshold, adjusting the PRI to the first PRI, and, when the result of the difference operation is greater than or equal to the threshold, maintaining the second PRI.

15. The ultrasound diagnosis method of claim 13, further comprising
scanning the contrast-enhanced image of the object by applying a second PRI with an increased pulse interval compared to a preset first PRI,
wherein the determining of the reflection artifact comprises:
obtaining third image data to which a third PRI, which is a PRI set when the contrast-enhanced image mode is set, has been applied;
obtaining second image data to which a second PRI having an increased pulse interval compared to the third PRI has been applied;
performing a difference operation between the third image data and the second image data; and
determining whether a result of the difference operation is greater than or equal to a preset threshold,
wherein the adjusting of the PRI comprises,
when the result of the difference operation is less than the threshold, adjusting the PRI to the third PRI, and, when the result of the difference operation is greater than or equal to the threshold, adjusting the PRI to the second PRI.
